(19) United States
(12) Patent Application Publication
(10) Pub. No.: US 2002/0197258 A1
(43) Pub. Date: Dec. 26, 2002

(54) Compositions and methods for preventing protein aggregation in neurodegenerative diseases

(76) Inventors: Hossein A. Ghanbari, Potomac, MD (US); Kasra Ghanbari, Potomac, MD (US)

Correspondence Address: Mary E. Gormley, Shaw Pittman LLP, 1650 Tysons Blvd., McLean, VA, 22102, US

(21) Appl. No.: 10/177,604

(22) Filed: Jun. 24, 2002

Related U.S. Application Data
(60) Provisional application No. 60/300,190, filed on Jun. 22, 2001.

(51) Int. Cl. A61K 39/395

(52) U.S. Cl. 424/146.1

(57) ABSTRACT

Disclosed are methods for treating a disease that involves protein aggregation, including Alzheimer's, Parkinson's, prion diseases such as BSE and CJD, and Down's syndrome. The methods involve administering to a subject suspected of having the disease a very high affinity antibody fragment immunoreactive with the protein that is aggregating. Such treatment will have the effect of preventing, slowing, or halting the disease progression by inhibiting protein aggregation.

This application claims the benefit of U.S. Provisional Application No. 60/300,190 filed Jun. 22, 2001, which is herein incorporated by reference in its entirety.

### BACKGROUND

1. Field of the Invention

The present invention is directed to a method of treating a disease that involves protein aggregation, comprising administering to a subject suspected of having said disease a very high affinity antibody immunoreactive with the protein that is aggregating. Such treatment will have the effect of slowing, or halting, the disease progression. Diseases where protein aggregation is causal or an associated symptom include, but are not limited to, Alzheimer's, Parkinson's, prion diseases such as BSE and CJD, and Down's syndrome.

2. Background of the Invention

A common manifestation of the onset or progression of many neurodegenerative disorders is the attraction of proteins into filaments in the brain, and the aggregation of these filaments into intracytoplasmic inclusions or extracellular plaque deposits. See Trojanowski and Lee, J. Alz. Disease, 3(1): 117-119 (2001), which is hereby incorporated by reference. An example of such filamentous lesions are the neurofibrillary tangles, composed of tau protein, and extracellular senile plaques composed of amyloid protein, which are seen in both Alzheimer's disease and Down's syndrome. Intraneuronal Lewy bodies, formed by the aggregation of α-synuclein, are seen in Parkinson's disease as well as Down's syndrome brains and other synucleinopathies. Prion diseases, such as Creutzfeld-Jakob disease (CJD), bovine spongiform encephalopathy (BSE), and scrapie, for instance, involve the conformational change and aggregation of prion proteins.

Recent therapeutic approaches to the treatment of these neurodegenerative diseases have focused on interfering with the aggregation of the lesion producing proteins. One such approach, for example, is the proposed immunization of patients with Alzheimer's disease with amyloid protein. Schenk et al. reported the observation that simple immunization of PDAPP transgenic mice with amyloid-forming peptide sequences both prevents plaque formation and ameliorates existent plaques in brain (Nature 400:173-7). (See also, WO072876A2 and WO072880A2.) PDAPP transgenic mice express a human amyloid precursor protein (APP) containing the FAD-associated V717F mutation under the control of the platelet-derived growth factor promoter. These mice show an age-dependent accumulation of extracellular amyloid plaques and an increase in astrocytosis. These results suggest that it might be possible to remove amyloid plaques from human brain if similar immunization worked in humans. Schenk et al. also showed that humoral-mediated (antibody-mediated) response, rather than T-cell mediated response, is the most likely mechanism by which A-beta immunization removes plaques from brain, which was shown by purifying IgG antibodies from A-beta-injected animals and re-injecting the purified antibodies into PDAPP transgenic animals at one-week intervals for six months. Amyloid burden was significantly reduced in the animals injected with IgG obtained from the mice injected with A-beta compared to animals injected with IgG purified from mice injected with an irrelevant antigen. Shenk et al. subsequently showed that monoclonal antibodies specific for A-beta could also reduce plaque burden when injected into PDAPP transgenic animals. The mechanism by which these anti-A-beta antibodies function appears to involve direct binding of antibody to amyloid within brain. The data suggest that a small amount of antibody must pass through the blood-brain barrier and enter the central nervous system. The use of antibody injection therapy to remove amyloid deposits in human brain may be important since it is well-established that the immune response and antibody repertoire are both reduced during aging. Thus, for elderly AD patients who do not elicit an effective immune response, the use of antibody injections may circumvent potential problems relating to this inability of patients to elicit an A-beta immune response.

Several obstacles to using antibodies therapeutically to treat diseases involving protein aggregation are envisioned, however. First, intact antibodies are so large as to pose problems in entering cells, as well as positioning themselves in between protein molecules that have formed filaments or plaques and breaking them apart. In addition, for treatment of humans, the antibodies should be humanized, so as to avoid an immune reaction to them. While such problems could be overcome by engineering a small, humanized, single chain fragment of the antibody, another issue to be dealt with is the fact that most antibodies have a rather low affinity, with a typical binding half-life on the order of seconds or minutes.

### SUMMARY OF THE INVENTION

The present invention overcomes the problems of the prior art by providing a method of treating neurodegenerative diseases by administering very high affinity, single chain antibody fragments with monovalent nanomolar to femtomolar antigen-binding affinity to the patient. Preferably, the antibody affinity is in the femtomolar range. The binding half-life of such antibodies is preferably more than about five days.

### DETAILED DESCRIPTION OF THE INVENTION

One object of the present invention is directed to a method of inhibiting the aggregation of a protein in a mammalian cell or tissue by adding to said cell or tissue a high affinity single chain antibody that is immunoreactive to the protein. By “inhibiting the aggregation of a protein” is meant interfering with a pathological protein aggregation, which is seen in several diseases, particularly what are referred to as neurodegenerative diseases. More preferably, the method is applicable to inhibiting aggregation of β-amyloid (or A-beta) (for Alzheimer's disease and Down's syndrome), α-synuclein (for synucleinopathies, such as Parkinson's disease), tau protein (e.g., for Alzheimer's and Down's syndrome), and prion protein (for prion diseases, such as CJD). Most preferably, the method is directed to treatment of Alzheimer's or Parkinson's disease by inhibiting the aggregation of β-amyloid or α-synuclein, respectively.

The method essentially involves the steric interference of the protein-protein interaction that leads to formation of protein filaments and/or the steric interference of the formation of the protein filaments into intracytoplasmic inclusions or extracellular plaques. In the present invention, protein aggregation is inhibited by a (very) high affinity, single chain antibody fragment that will bind to the protein in question, either in or outside the cell, and thereby sterically hinder any intra- or intermolecular interaction. This mode of action is in contrast to other recent proposals involving passive immunization with anti-β-amyloid antibodies, which are based on interaction with extracellular plaques and clearance by microglial cells (although the present invention does not preclude this additional action). See, e.g., Bacskai, B. J. et al., Nature Medicine, Vol. 7, No. 3, pp. 369-372 (March, 2001).

The antibodies referred to above may be engineered from single chain Fv fragments of known monoclonal antibodies by the methods described in Boder et al., PNAS, 97(20):10701-10705 (2000), and the associated commentary of Foote and Eisen, PNAS, 97(20):10679-10681 (2000), both of which are hereby incorporated by reference. The method uses affinity maturation of antibody fragments to produce in vitro Fv fragments with equilibrium constants as high as the femtomolar range with slow dissociation kinetics (half-life>5 days). The directed evolution of these fragments results in affinities that are not attainable in vivo; affinity maturation in B cells exhibits an apparent affinity ceiling in the nanomolar range.

In order to obtain the very high affinity mutants of scFv antibodies, one need only start with a monoclonal antibody to the protein in question, from which an scFv fragment is obtained by well known methods. Monoclonals to the various proteins that are involved in neurodegenerative diseases are known and/or commercially available, but also could be made using existing technology. For instance, U.S. Pat. No. 6,238,892, which is incorporated herein by reference, discloses monoclonal antibodies to the tau protein, which is involved in Alzheimer's and Down's syndrome, among others. Monoclonals reactive with amyloid-P protein, which is involved in Alzheimer's disease, among others, is disclosed in U.S. Pat. No. 5,786,180, which is hereby incorporated by reference, and are also commercially available from Alpha Diagnostic International, Inc., San Antonio, Tex.). Antibodies reactive with PrP^{SC} and PrP^{C} are disclosed in U.S. Pat. Nos. 4,806,627 and 6,214,565, which are hereby incorporated by reference and are also commercially available from Chemicon (Temecula, Calif.). Monoclonal antibody to α-synuclein is commercially available from Transduction Laboratories (subsidiary of Becton Dickinson) (CA). In addition to the specific antibodies disclosed in the above referenced patents, the methods of producing the same can also be used to generate other monoclonal antibodies, and the present invention is not so limited. Furthermore, techniques to humanize the antibodies, if necessary, are well known in the art. Humanization can be essentially performed following the method of Winter and co-workers (Jones et al., Nature 321:522 (1986); Riechmann et al., Nature 332:323 (1988); and Verhoeyen et al., Science 239:1534 (1988)), by substituting rodent CDRs for CDR sequences for the corresponding sequences of a human antibody.

The particular epitope with which the monoclonal reacts is not critical in the present invention, because it is the steric hindrance that is of concern in this methodology. For instance, there is presumably no function of the β-amyloid protein, which is derived by cleavage of the much larger amyloid precurser protein. Therefore, intereference in the function is not a concern. This is in contrast to the disclosure of Solomon, U.S. Pat. No. 5,688,651, hereby incorporated by reference, which considers it essential that the monoclonal antibody not interfere with bioactivity of the aggregating protein.

What is essential to the present invention is that the scFv's have a very high affinity (low dissociation constant) to the target protein, which will ensure that the fragment stays bound to the antigen long enough to exert its influence in preventing or inhibiting aggregation. The Fv antibody fragments useful in the present invention are those with equilibrium constants above the nanomolar range, and preferably as high as the femtomolar range with slow dissociation kinetics (half-life>5 days).

In accordance with the methods of Boder et al., supra, a library of randomly mutated scFv's is constructed using the sexual PCR method of Stemmer (Stemmer, W. P., Nature, 370, pp.389-391, 1994), and transforming the library DNA into yeast by the method of Geitz et al. (http://tto.trends.com), by which the recombinant DNA is fused to the AGA2 gene of S. cerevisiae. The AGA2 fusion protein is secreted and attaches to the surface of the yeast cell. The yeast display the mutagenized scFv's on their surface, and those clones exhibiting increased antibody-antigen dissociation kinetic constants with fluoroscein-labelled antigen are identified and isolated by flow cytometry.

Delivery of the therapeutic antibody fragments of the present invention raises two obstacles: delivery to the affected brain tissue; and intracellular delivery in diseases where the aggregated protein is primarily intracellular. The first obstacle can be overcome by nasal administration of the antibody fragment, and is the preferred route for the treatment of neurodegenerative diseases, because it will allow the agent to get directly to the brain. Nasal administration can be in the form of a liquid spray or a powder spray, a gel, ointment, infusion, injection, or nose drops. Liquid or powder sprays are preferred. The agent is inhaled through the nasal passages and absorbed by the nasal mucosa, where in turn the agent will travel through the olfactory neural pathway to the brain.

Other methods to administer the scFv's would include directly infusing into the cerebrospinal fluid or brain parenchyma. The diffusion of the scFv's into the tissue can be supplemented by the convection-enhanced delivery of macromolecules developed by Oldfield and colleagues (see Bobo et al., PNAS USA, 91: 2076-2080 (1994); Lonser et al., J. Neurosurg, 91:294-302 (1999); Chen et al., J. Neurosurg., 90:315-320 (1999); Morrison et al., Am. J. Physiol., R1218-R1229; and Zirzow et al., Neurochem. Res., 24(2):301-305 (1999); each of the foregoing of which are hereby incorporated by reference), which relies on maintaining a pressure gradient during interstitial infusion. The use of mannitol also allows increased delivery of intraventricularly administered agents (see Ghodsi et al., Experimental Neurol., 160:109-116 (1999); and Mastakov et al., Mol. Therapy, 3(2):225-232 (2001), each of which is hereby incorporated by reference). For further methods, see generally Raymond T. Bartus, Current Opinion in Drug Discovery & Development, 2(2):152-166, which is hereby incorporated by reference.

While extracellular aggregation products can be disrupted, or their formation inhibited, by delivering the antibody fragment directly to the affected tissue area, intracellular filaments or aggregated proteins require the intracellular delivery of the antibody fragment. This may require more sophisticated delivery systems, several of which are known in the art. For instance, the antibody fragments can be encased in liposomes for intracellular delivery by fusion with the targeted cells. One can also recombinantly express the very high affinity antibody fragment in vivo. This method comprises the intracellular expression of an antibody capable of binding to the protein aggregation target. A DNA sequence (referred to as an “antibody cassette”), containing a sufficient number of nucleotides coding for the Fv portion of an antibody capable of binding to the target (such as amyloid protein, synuclein, tau, etc.) operably linked to a promoter that will permit expression of the antibody in the cell(s) of interest, is delivered to a cell. Thereafter, the antibody is expressed intracellularly and binds to the target, thereby preventing further aggregation or disrupting aggregated protein. This antibody is sometimes referred to as an “intrabody”. Such methods are analogous to those described in U.S. Pat. Nos. 6,004,940 and 6,143,520, which are hereby incorporated by reference.

Still another way to deliver the very high affinity antibody fragment intracellularly is by chemically or recombinantly (covalently or noncovalently) attaching it to a modified toxin that has membrane penetrating properties. An example is exotoxin A of Pseudomonas (or ETA). This toxin has been extensively studied, and the portion of the toxin that is responsible for receptor binding and membrane penetration is known (domains I and II). Domain III contains the toxic enzymatic activity. The toxin is modified such that domain III is replaced by the very high affinity, single chain Fv of the present invention. Methods for accomplishing this are described in U.S. Pat. No. 6,086,900, which is hereby incorporated by reference. Another, more preferred, toxin to use in this method is diphtheria toxin, because it preferentially binds to receptors on neuronal cells. This toxin contains two fragments, A and B; A is responsible for the toxic enzymatic activity and B is responsible for receptor binding and membrane penetration. In an analogous fashion, fragment A would be replaced with the very high affinity antibody fragment for intracellular delivery via the receptor binding of B.

Compositions containing a very high affinity, single chain antibody in a pharmaceutically acceptable medium are also one aspect of the present invention. A composition may contain more than one antibody fragment to either the same or different target proteins. For instance, in the treatment of Alzheimer's disease, an scFv to β-amyloid and an scFv to tau protein. Another aspect of the present invention is a therapeutic composition comprising a gene delivery system, which produces intracellular antibody fragment as described above. Pharmaceutically acceptable media are biologically compatible vehicles which are suitable for administration to an animal: e.g., physiological saline. A therapeutically or prophylactically effective amount of a compound is an amount which is capable of producing a medically desirable result such as reduced or prevented protein aggregation of a targeted protein in a treated animal, preferably human.

The compositions can be administered for prophylactic and/or therapeutic treatment of diseases related to the aggregation of proteins in the brain. In therapeutic applications, the pharmaceutical compositions are administered to a host already suffering from the disease. The pharmaceutical compositions will be administered in an amount sufficient to inhibit further aggregation of the disease protein. An amount adequate to accomplish this defined as a “therapeutically effective dose.” Such effective dose will depend on the extent of the disease, the size of the host, and the like, but will generally range from about 0.1 μg to 10 mg of the compound per kilogram of body weight of the host, with dosages of 0.1 μg to 1 mg/kg being more commonly employed. The frequency of administration would depend on how an individual subject responds to the treatment, but could generally be weekly or monthly or more because of the slow dissociation of the antibody fragments. It is contemplated that the therapy will be continued perhaps for the life of the subject.

For prophylactic applications, the pharmaceutical compositions of the present invention are administered to a host susceptible to the various protein aggregation-related neurodegenerative diseases, but not already suffering from such disease. Such hosts may be identified by genetic screening and clinical analysis, as described in the medical literature (e.g. Goate (1991)Nature 349:704-706). The pharmaceutical compositions will be able to inhibit or prevent aggregation of the protein at a symptomatically early stage, preferably preventing even the initial stages of onset. The amount of the compound required for such prophylactic treatment, referred to as a prophylactically effective dosage, is generally the same as described above for therapeutic treatment.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed:

1. A method of inhibiting the aggregation of a protein in a mammalian cell or tissue, comprising adding to said cell or tissue a high affinity single chain antibody fragment that is immunoreactive to said protein, wherein the antibody fragment has a dissociation constant in the nanomolar to femtomolar range.

2. The method of claim 1, wherein said protein is amyloid protein.

3. The method of claim 1, wherein said protein is α-synuclein.

4. The method of claim 1, wherein said protein is tau protein.

5. The method of claim 1, wherein said protein is PrP.

6. The method of claim 1, wherein said mammal is a human.

7. The method of claim 1, wherein the dissociation constant is in the femtomolar range.

8. The method of claim 1, comprising administering to a subject suspected of having Alzheimer's disease a high affinity antibody fragment immunoreactive with one of β-amyloid and tau protein, wherein the antibody fragment has a dissociation constant in the nanomolar to femtomolar range and wherein said antibody fragment will inhibit the aggregation of the corresponding β-amyloid or tau antigen.

9. The method of claim 1, comprising administering to a subject suspected of having Parkinson's disease, a high affinity antibody immunoreactive with α-synuclein, wherein the antibody fragment has a dissociation constant in the nanomolar to femtomolar range and wherein said antibody fragment will inhibit the aggregation of the α-synuclein.

10. The method of claim 1, comprising administering to a subject suspected of having a prion-associated disease, a high affinity antibody immunoreactive with PrP protein, wherein the antibody fragment has a dissociation constant in the nanomolar to femtomolar range and wherein said antibody fragment will inhibit the aggregation of the PrP.

11. A composition comprising one or more high affinity antibody fragments immunoreactive with one or more of β-amyloid protein, tau protein, α-synuclein and PrP protein in admixture with a pharmaceutically acceptable medium, wherein the antibody fragment, or fragments, has a dissociation constant in the nanomolar to femtomolar range.

12. The composition of claim 11, a high affinity antibody, wherein the fragment is immunoreactive with tau protein.

13. The composition of claim 11, wherein the high affinity antibody fragment is immunoreactive with α-synuclein.

14. The composition of claim 11, high affinity antibody, wherein the fragment is immunoreactive with PrP protein.

15. The composition of claim 11, which comprises an antibody fragment immunoreactive to β-amyloid and an antibody fragment immunoreactive to tau protein.
